(19) United States
(12) Patent Application Publication
(10) Pub. No.: US 2024/0342316 A1
(43) Pub. Date: Oct. 17, 2024

(54) A PHARMACEUTICAL COMPOSITION FOR IMAGING

(71) Applicant: ASTELLAS PHARMA INC., Tokyo (JP)

(72) Inventors: Masako Saito, Tokyo (JP); Kanji Komatsu, Tokyo (JP); Shin Takusagawa, Tokyo (JP); Leticia Delgado-Herrera, Lake Forest, IL (US)

(21) Appl. No.: 18/291,487

(22) PCT Filed: Jul. 27, 2022
(86) PCT No.: PCT/IB22/56957

§ 371 Date: Jan. 23, 2024

Related U.S. Application Data
(60) Provisional application No. 63/226,534, filed on Jul. 28, 2021.

(51) Int. Cl. A61K 49/00

(52) U.S. Cl. CPC A61K 49/0034; A61K 49/0054

(57) ABSTRACT

Intraoperative ureter identification helps reduce the risk of ureteral injury. In this first-in-human phase 1 study, Pudexacianinium chloride was administered intravenously in healthy adult participants as a single bolus dose. This invention is related to a new amount of administration using Pudexacianinium or pharmaceutically acceptable salt thereof for intraoperative NIRF ureter visualization.

### FIELD

The present invention relates to a pharmaceutical composition comprising a suitable amount of Pudexacianinium chloride for imaging at least one of an organ, a body fluid, and a vessel in a living body by near-infrared fluorescence or a method for administering the pharmaceutical composition.

### BACKGROUND

Although ureteral injury is an infrequent occurrence, 75% of all cases occur during abdominal or pelvic surgery,¹ largely as a consequence of the close proximity of the ureter to anatomical structures encountered during the procedure.² Most iatrogenic ureteral injuries (IUIs) stem from gynecological procedures,³ where rates of 0.1% to 1.5% have been reported for non-oncologic surgeries.⁴ Colorectal surgery is the second most common source of IUIs. An analysis of more than 2 million colorectal surgeries performed in the United States over 10 years identified a 0.28% rate of IUIs, representing 6,027 injuries.⁵ If not detected and treated promptly, IUIs can have serious sequelae, increasing morbidities such as ureteral strictures and reduced long-term renal function,⁶ and contributing to longer hospital stays, increased hospital costs, and increased mortality.⁵ In addition, IUIs are not without medicolegal and financial implications for the surgeon.⁷ For IUIs, the single greatest prognostic factor is time to diagnosis; superior outcomes are associated with intraoperative diagnosis and repair.^{4,8}

Detection of IUI, however, can be difficult, and only about one-third of IUIs are diagnosed intraoperatively.¹⁸⁻¹⁰ Prevention of IUI—by far the most desirable course—is often stymied by the challenges involved in ureter identification, particularly during laparoscopic procedures.¹¹⁻¹³ Prophylactic ureteral stenting, which aids in ureter visualization, is sometimes employed in complex procedures,¹⁴ but remains controversial and has the potential itself to induce IUIs.⁵ Although considered acceptable in high-risk procedures, current guidelines do not call for its routine use.^{8, 15} Preoperative imaging techniques such as intravenous (IV) urography and computed tomography can be used, but do not provide real-time visualization and may not prevent IUIs.⁸ Clearly, better noninvasive methods of ureter identification are needed and 1 survey even found that the majority of surgeons (54.5%) would consider implementing such a technique in their regular daily practice.¹⁴

Near-infrared fluorescence (NIRF) imaging is a promising technique for real-time visualization of anatomical structures.^{2, 12, 16-19} Preoperative injection of a renally excreted NIRF contrast agent that can be detected by intraoperative imaging systems allows for real-time ureter visualization and avoidance without the use of radionuclides. NIR light can penetrate through 5 millimeters of tissue,¹⁸ provide a strong visual signal due to low tissue autofluorescence and weak absorption in the NIR range,¹⁸ and does not change the visual appearance of the surgical field, thereby providing “an enhanced reality beyond standard white light visual inspection and palpitation.”¹² Key to these desirable properties, however, is the contrast agent itself. The first-in-human study of this technique for ureter identification used the dye, methylene blue,¹⁹ but this agent did not provide sufficient optical properties to offer any visualization advantage with NIR over white light.¹⁶ Improvements in NIFR contrast agents for ureter visualization has consequently been an area of active research.¹²

Thus, a need exists for NIFR contrast agents to assist in identification of ureter identification.

### SUMMARY

Provided herein are pharmaceutical compositions comprising Pudexacianinium or a pharmaceutically acceptable salt thereof for imaging at least one of an organ, a body fluid, and a vessel in a subject by near-infrared fluorescence (NIRF), wherein the pharmaceutical composition comprises 0.3 mg to 24.0 mg of Pudexacianinium as a free form and the pharmaceutical composition is administered to the subject in a first administration and optionally the pharmaceutical composition comprising 0.3 mg to 24.0 mg of Pudexacianinium as a free form is re-administered to subject after the first administration. In some embodiments, the pharmaceutical composition is re-administered to the subject after the first administration. In some embodiments, the Pudexacianinium or a pharmaceutically acceptable salt thereof is Pudexacianinium chloride. In various embodiments, the pharmaceutical composition comprises 0.3 mg to 3.0 mg of Pudexacianinium as a free form. In some embodiments, the pharmaceutical composition comprises 1.0 mg to 3.0 mg of Pudexacianinium as a free form.

Further provided herein are methods for imaging at least one of an organ, a body fluid, and a vessel in a subject by near-infrared fluorescence (NIRF) comprising administering to the subject a first pharmaceutical composition comprising Pudexacianinium or a pharmaceutically acceptable salt thereof at a dosage of 0.3 mg to 24.0 mg of Pudexacianinium free form; subjecting the subject to NIRF to obtain an image of the at least one of an organ, a body fluid, and a vessel in the subject; and optionally administering to the subject a subsequent pharmaceutical composition comprising Pudexacianinium or a pharmaceutically acceptable salt thereof at a dosage of 0.3 mg to 24.0 mg of Pudexacianinium free form. In some embodiments, the first pharmaceutical composition comprises Pudexacianinium or a pharmaceutically acceptable salt thereof at a dosage of 0.3 mg to 3.0 mg of Pudexacianinium free form, or the subsequent pharmaceutical composition comprises Pudexacianinium or a pharmaceutically acceptable salt thereof at a dosage of 0.3 mg to 3.0 mg of Pudexacianinium free form, or both. In various embodiments, the first pharmaceutical composition comprises Pudexacianinium or pharmaceutically acceptable salt thereof at a dosage of 1.0 mg to 3.0 mg of Pudexacianinium free form, or the subsequent pharmaceutical composition comprises Pudexacianinium or pharmaceutically acceptable salt thereof at a dosage of 1.0 mg to 3.0 mg of Pudexacianinium free form, or both. In some embodiments, Pudexacianinium or pharmaceutically acceptable salt thereof is Pudexacianinium chloride.

Further provided herein are use of Pudexacianinium or a pharmaceutically acceptable salt thereof for the manufacture of a medicament for imaging at least one of an organ, a body fluid, and a vessel in a subject by near-infrared fluorescence wherein said medicament comprises 0.3 to 24.0 mg of Pudexacianinium as free form at the first administration and optionally at the second administration after the first administration. In some embodiments, the medicament comprises 0.3 to 3.0 mg of Pudexacianinium as free form. In various embodiments, the medicament comprises 1.0 mg to 3.0 mg of Pudexacianinium as free form. In some embodiments, Pudexacianinium or pharmaceutically acceptable salt thereof is Pudexacianinium chloride.

### BRIEF DESCRIPTION OF THE DRAWINGS

A more complete appreciation of the disclosure and many of the attendant advantages thereof will be readily obtained as the same become better understood by reference to the following detailed description when considered in connection with the accompanying drawings.

FIG. 1 is the study consisting of a screening period, an investigational period, and a follow-up period for assessment of Pudexacianium as a NIFR contrast agent.

FIG. 2A is Pudexacianinium arithmetic mean plasma concentrations.

FIG. 2B is Urinary Pudexacianinium concentrations which were quantifiable for up to 6 hours postdose in the 0.1-mg and 0.5-mg cohorts, and for up to 24 hours postdose in the 2-mg, 8-mg, and 24-mg cohorts.

FIG. 3A is at doses ranging from 0.1 mg to 24 mg, increases in Pudexacianinium AUC_{inf}.

FIG. 3B is at doses ranging from 0.1 mg to 24 mg, increases in Pudexacianinium Cₘₐₓ.

FIG. 4 is the simulated urine Pudexacianinium concentration-time courses in patients under anesthesia during surgery.

FIG. 5 is the simulated proportions of patients target urine concentration (1 μg/mL).

### DETAILED DESCRIPTION

Pudexacianinium is a novel indocyanine green (ICG) derivative containing 3-cyclodextrin moieties.²⁰ Its molecular size and hydrophilic nature allow for its excretion into urine, imparting a visibly green coloration and, with much greater sensitivity, enabling ureteral-specific imaging and visualization using existing near-infrared ICG detection devices.²¹ Preclinical results showed that IV Pudexacianinium as free form at 0.01 mg/kg allowed visualization of ureters for up to 3 hours postadministration.²² Briefly, the proportions of animals whose ureters were visible up to 3 hours after administration of Pudexacianinium chloride were 33% at 0.001 mg/kg and 100% at 0.01 mg/kg, respectively. In addition, Pudexacianinium chloride was well tolerated, having no toxicity-related changes when dosed once daily for 4 weeks of up to 300 mg/kg (as free form) in cynomolgus monkeys.²²

Pudexacianinium as a free form is a cation form having the following structure:

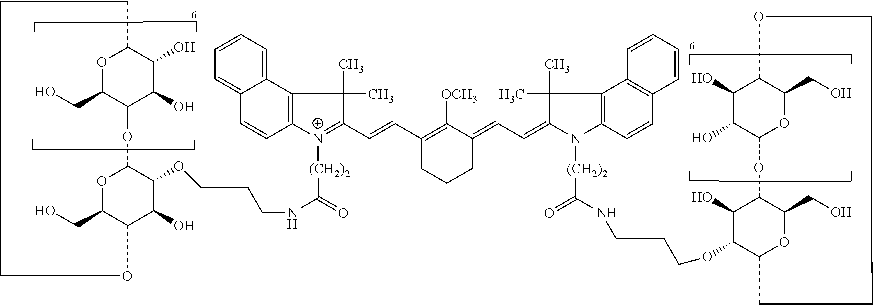

and it can be provided as a salt form with a coordinating anion, e.g., which is generated by removing one or more proton from an acid, e.g., chloride to provide a Pudexacianinum chloride salt. Examples of such salts include salts with a coordinating anion derived from inorganic acids such as hydrochloric acid, hydrobromic acid, hydroiodic acid, sulfuric acid, nitric acid, phosphoric acid, and the like, and with a coordinating anion derived from organic acids such as formic acid, acetic acid, propionic acid, oxalic acid, malonic acid, succinic acid, fumaric acid, maleic acid, lactic acid, malic acid, mandelic acid, tartaric acid, dibenzoyl tartaric acid, ditoluoyl tartaric acid, citric acid, methanesulfonic acid, ethanesulfonic acid, benzenesulfonic acid, p-toluenesulfonic acid, aspartic acid, glutamic acid, and the like.

A phase 1 study of Pudexacianinium chloride evaluated the safety and tolerability, as well as the pharmacokinetics (PK) of a single dose of Pudexacianinium chloride administered to healthy human volunteers. A suitable dose of Pudexacianinium as free form has been investigated for administering to a subject based on the preclinical results and phase 1 study from multiple pharmacological viewpoints.

As a result, certain doses of Pudexacianinium as a free base has been discovered as a suitable dose for imaging at least one of an organ, a body fluid, and a vessel in a living body by near-infrared fluorescence. The term “suitable dose” means an amount when administered to the subject which results in beneficial or desired results, including clinical results, e.g., safety dose or efficacy dose for imaging at least one of an organ, a body fluid, and a vessel in a living body by near-infrared fluorescence. In some embodiments, the dosage may be in the range from about 0.3 to 5.0 mg per subject, based upon Pudexacianinium free form weight. In some embodiments, the dosage may be in the range from about 0.3 to 3.0 mg per subject, based upon Pudexacianinium free form weight. In some embodiments, the dosage may be in the range from about 0.3 to 1.0 mg per subject, based upon Pudexacianinium free form weight. In some embodiments, the dosage may be in the range from about 1.0 to 3.0 mg per subject, based upon Pudexacianinium free form weight. In some embodiments, the dosage may be 0.3, 0.5, 1.0, 1.5, 2.0, 2.5, 3.0, 3.5, 4.0, 4.5, or 5.0 mg/subject, based upon Pudexacianinium free form weight. In some embodiments, the dosage may be 0.3, 0.5, 1.0, 1.5, 2.0, 2.5, or 3.0 mg/subject, based upon Pudexacianinium free form weight. In some embodiments, the dosage may be 0.5, 1.0, 1.5, 2.0, 2.5, or 3.0 mg/subject, based upon Pudexacianinium free form weight. In some embodiments, the dosage may be 1.0, 1.5, 2.0, 2.5 or 3.0 mg/subject, based upon Pudexacianinium free form weight. In some embodiments, the dosage may be 1.0 or 3.0 mg/subject, based upon Pudexacianinium free form weight. In some embodiments, the dosage may be 1.0 mg/subject, based upon Pudexacianinium free form weight. In some embodiments, the dosage may be 3.0 mg/subject, based upon Pudexacianinium free form weight. The dosage for the first administration and the dosage for the second administration may be same or different and may be any combination of the dosage described above. In some embodiments, the dosage for the first administration and the dosage for the second administration may be same.

In particular, disclosed herein are pharmaceutical compositions comprising Pudexacianinium or a pharmaceutically acceptable salt thereof (e.g., chloride) for imaging at least one of an organ, a body fluid, and a vessel in a subject by near-infrared fluorescence, wherein said pharmaceutical composition comprising 0.3 mg to 24.0 mg of Pudexacianinium as a free form is administered to subject first and optionally 0.3 mg to 24.0 mg of Pudexacianinium as a free form is re-administered to subject after the first administration. Also disclosed herein are methods for imaging at least one of an organ, a body fluid, and a vessel in a subject by near-infrared fluorescence comprising administering 0.3 to 24.0 mg of Pudexacianinium as free form to subject first and optionally re-administering 0.3 to 24.0 mg of Pudexacianinium as free form to subject after the first administration. Further provided herein are uses of Pudexacianinium or a pharmaceutically acceptable salt thereof (e.g., chloride) for the manufacture of a medicament for imaging at least one of an organ, a body fluid, and a vessel in a subject by near-infrared fluorescence wherein said medicament comprises 0.3 to 24.0 mg of Pudexacianinium as free form at the first administration and optionally at the second administration after the first administration.

The terms “administer”, “administering”, “administration”, and the like, as used herein, refer to methods that may be used to enable delivery of compositions to the desired site of biological action. These methods include, but are not limited to, intraarticular (in the joints), intravenous, intramuscular, intradermal, intraperitoneal, subcutaneous, topically, and the like. In some embodiments, the administration is intravenous administration.

The Pudexacianinium, e.g., Pudexacianinium chloride, can be administered as a pharmaceutical composition in the methods disclosed herein. Pharmaceutical composition may include a pharmaceutically acceptable carrier and additive according to the administration. The types of a pharmaceutically acceptable carrier and additive are not particularly limited, but a carrier and an additive well known to those skilled in the art can be used. In some embodiments, the pharmaceutical composition is a solution, e.g., a water solution for administration. Concentration of Pudexacianinium as free form in water solution composition may be in the range of about 0.1 to. 8.0 mg/mL. In some embodiments, the concentration may be in the range of 0.5 to 4.0 mg/mL. In some embodiments, the concentration may be in the range of 1.0 to 4.0 mg/mL. In some embodiments, the concentration may be 0.5, 1.0, 2.0, 3.0 or 4.0 mg/mL. In some embodiments, the concentration may be 1.0 mg/mL. In some embodiments, the concentration may be 2.0 mg/mL. In some embodiments, the concentration may be 3.0 mg/mL. In some embodiments, the concentration may be 4.0 mg/mL.

In some embodiments, the timing of the administration of the pharmaceutical compositions disclosed herein is before an operation (e.g., before laparoscopic surgery) or intraoperation (e.g., during laparoscopic surgery). In some embodiments, a first administration is before operation and optionally further comprises a second administration of the pharmaceutical composition, such as re-administering it intraoperation.

In order to detect Pudexacianinium for a diagnosis the following device can be used: The device is a device used for measuring at least a part of a living body to which the diagnostic composition of the present invention described above is administered (See U.S. Pat. No. 9,056,131, the disclosure of which is incorporated by reference in its entirety).

### EXAMPLES

Herein below, the disclosed methods and compositions are described in more detail with reference to Working Examples. Further, the present disclosure is not limited to the following Examples.

### Example 1—Rationale for Dose Levels

The starting dose was 0.1 mg of Pudexacianinium as free form per subject. The rationale for the starting dose was based on the results of the toxicology studies in cynomolgus monkeys and the estimated clinical efficacious dose in humans.

The estimated clinical efficacious dose is 0.5 mg of Pudexacianinium as free form per subject. In the ex vivo imaging study of isolated pig ureter using Pudexacianinium chloride as a NIR-F agent,¹ sufficient ureteral visualization was defined as the ureter was sufficiently noted visually in the captured images under fluorescent imaging at 1 μg/mL. In addition, in the imaging study of minipigs,¹ the ureter was visually identifiable under fluorescent imaging at a urinary concentration greater than 1 μg/mL at 3 hours after intravenous administration of 0.01 mg/kg Pudexacianinium as free form. These observations support the clinical efficacious dose chosen in the study, permitting sufficient intraoperative visualization of the ureter for up to 3 hours, which exceeds the typical length of a routine surgical procedure (approximately 2 hours). Therefore, the estimated clinical efficacious dose was calculated as the dose that gives urine concentration above 1 μg/mL at 3 hours after intravenous administration of Pudexacianinium with body-weight correction using the following equation:

Estimated clinical efficacious dose=0.01 mg/kg(animal dose)×(40 kg [animal weight]/60 kg [human weight])^{0.33}×60 kg(human weight)=0.5 mg/subject

Criteria for handling concentrations below the limit of quantification in pharmacokinetic analysis:

Concentration values that were below the level of quantification (BLQ) were set to zero, with defined exceptions as follows:

Any embedded BLQ value (between two quantifiable concentrations) and BLQ values following the last quantifiable concentration in a profile were set to missing for the purposes of pharmacokinetic (PK) analysis.

If there were late positive concentration values following two BLQ concentration values in the apparent terminal phase, these values were evaluated. If these values were considered to be anomalous, they were set to missing.

If an entire concentration-time profile was BLQ, the profile was excluded from the PK analysis.

If a predose concentration was missing, these values were set to zero by default in Phoenix WinNonlin.

### Study Design:

The study (ClinicalTrials.gov Identifier: NCT03698305) was a randomized, double-blind, placebo-controlled, sequential ascending IV bolus dose group study conducted at a single center (Covance Clinical Research Unit, Inc.) in the United States. The objectives were to assess the safety and tolerability of Pudexacianinium chloride administered IV as a single dose to healthy participants, and to assess the single-dose PK profile of Pudexacianinium in plasma and urine.

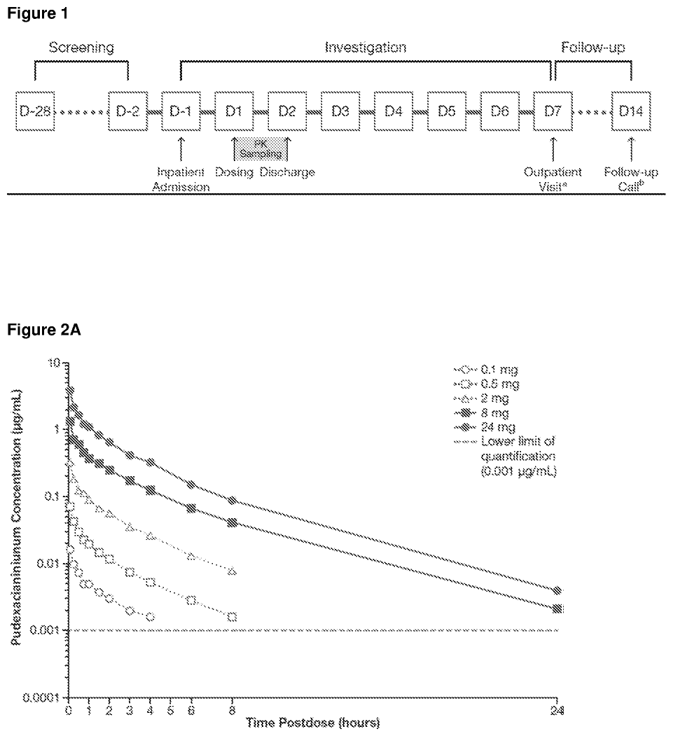

The study population, 30 participants in all, was comprised of 5 cohorts consisting of 6 healthy volunteers (3 females and 3 males) per cohort. Participants were randomly assigned 2:1 (n=4 and n=2 in each cohort) to receive a single IV bolus dose of Pudexacianinium chloride or placebo. The study consisted of a screening period, an investigational period, and a follow-up period (FIG. 1). Following successful screening, participants were admitted to the clinic on study day −1. On day 1, participants had an indwelling urethral catheter placed 1-2 hours before dosing which remained until ≥8 hours postdose. Under fasting conditions, participants in Cohort 1 randomized to Pudexacianinium received 0.1 mg of Pudexacianinium as free form by IV bolus; subsequent cohorts received 0.5-mg, 2-mg, 8-mg, and 24-mg of Pudexacianinium as free form boluses, sequentially. The estimated clinical efficacious dose for Pudexacianinium as free form is 0.5 mg per subject. In the imaging study of Gottingen minipigs, the ureter under fluorescent imaging was visually identifiable at more than 1 μg/mL of urinary concentration at 3 hours after IV administration of Pudexacianinium as free form.²² The efficacious dose in humans, therefore, was calculated as the dose that gives urine concentration above 1 μg/mL at 3 hours after IV administration of Pudexacianinium chloride. To evaluate the dose dependency of Pudexacianinium chloride PK in the first-in-human study, the starting dose of 0.1 mg, which is fivefold lower than the estimated clinical efficacious dose (0.5 mg of Pudexacianinium as free form), was selected.

After at least 5 of 6 patients in a cohort had completed the study procedure, a Dose-Escalation Committee reviewed all PK and safety data, and decided whether to proceed with dose escalation, stop dose escalation, repeat a dose level, or investigate a lower dose level intermediate between the current and prior doses. Pudexacianinium chloride solution for injection was supplied as a 4-mg/mL of Pudexacianinium as free form aqueous solution for IV injection. Pudexacianinium chloride solution was provided in 10 mL amber glass vials.

Blood and urine samples were collected over a 24-hour period. Participants were discharged on day 2, returned to the clinic for a follow-up visit on day 7, and the study was completed with a follow-up phone call on day 14. To prevent accidental unblinding should the urine be discolored, catheter and tubing collection bags were covered, and participants were prevented from observing these or the collection vials when changed. Collection and processing of urine samples was conducted by a separate, additional unblinded staff that did not participate in the assessments.

Informed consent was obtained from each participant and the protocol was approved by the Institutional Review Board (Advarra IRB, Columbia, MD, USA). The study was further conducted in accordance with the principles of the Declaration of Helsinki, Good Clinical Practice, and International Council for Harmonisation of Technical Requirements for Pharmaceuticals for Human Use guidelines. Amendments made to the study protocol, discussed below (see “Participants” and “Assessments”), did not affect outcomes in this study.

Pudexacianinium chloride is used as follows:

Molecular weight 3079.44 (chloride salt) 3043.99 (free form)
Storage conditions Store in a freezer (−80° C., actual temperature: −81.5° C. to −74.6° C.),
Protection from light, Filled with nitrogen gas
   Usage Relative humidity of 10% or lower

### Participants:

Eligible participants were 18-55 years of age, with a body mass index (BMI) of 18.5 to 32.0 kg/m², inclusive, and weighing >40 kg (females) or >50 kg (males). Female participants were excluded if pregnant and were required to abstain from breastfeeding throughout the treatment period and for at least 30 days after final drug administration. All participants were required to follow contraceptive guidelines. All participants had normal liver function (alanine aminotransferase, aspartate aminotransferase, alkaline phosphatase, gamma-glutamyl transferase, and total bilirubin <upper limit of normal [ULN]) and normal renal function (blood urea nitrogen and creatinine ≤ULN) at day −1, the latter added as an amendment to the protocol on Oct. 31, 2018. A history or evidence of clinically significant disease or malignancy was not permitted. All participants provided written informed consent.

### Assessments:

Safety and tolerability were assessed at each dose level through monitoring of adverse events (AEs) associated with Pudexacianinium chloride, clinical laboratory tests, vital signs, electrocardiograms (ECGs), and physical examinations. Endpoints for safety and tolerability were the nature, frequency, and severity of AEs and clinical laboratory tests, vital signs, routine 12-lead ECGs, and physical examinations. If subjects developed a hypersensitivity reaction, an additional blood sample for determination of histamine concentration was taken as soon as possible after the onset of the hypersensitivity reaction.

Adverse events were graded according to the National Cancer Institute—Common Terminology Criteria for Adverse Events, version 5.0, as specified in the Oct. 31, 2018 protocol amendment. Adverse events were categorized by organ class and preferred term using the Medical Dictionary for Regulatory Activities, version 21.1. Green coloration of the urine was not considered an AE as this was an expected and known reversible effect lacking any untoward clinical symptoms.

Timepoints for blood and urine PK sample collection are shown (Table 1). Urine interval sampling was between −1 hour and time of dosing, and between each subsequent consecutive urine sample collection time point. Endpoints for PK parameters of Pudexacianinium in plasma were back-extrapolated plasma concentration at time zero (C₀); maximum observed plasma concentration (Cₘₐₓ); area under the plasma concentration-time curve from time zero to 24 hours postdose (AUC₀₋₂₄), from time zero to time of last quantifiable concentration (AUCₗₐₛₜ), from time zero to infinity (AUC_{inf}), and from time of last quantifiable concentration to infinity as percentage of total area under the concentration-time curve (AUC_{inf}[% extrap]); total body clearance of drug from plasma (CLₜₒₜ), time of maximum observed concentration (tₘₐₓ); apparent terminal elimination half-life (t_{1/2}); and volume of distribution during the terminal phase (Vz). Endpoints for PK parameters of Pudexacianinium measured in urine were amount of unchanged drug excreted into the urine (Ae), percentage of dose excreted into the urine (Ae %), cumulative amount of unchanged drug excreted into the urine (CumAe), percentage of CumAe (CumAe %), amount of unchanged drug excreted into the urine from time zero to the time of last quantifiable concentration (Aeₗₐₛₜ), percentage of Aeₗₐₛₜ (Aeₗₐₛₜ%), renal clearance (CL_{R}), and mean Pudexacianinium urine concentration at each time point.

Start and stop interval times of green urine coloration were recorded for the 8-mg and 24-mg Pudexacianinium as free form cohorts per the Apr. 17, 2019 protocol amendment. Detection of coloration in the urine was performed by unblinded nursing staff using visual inspection only. Coloration records were maintained in a secure location until post database lock to avoid potential unblinding of blinded staff.

### Statistical Methods:

The safety population comprised all participants who received a single dose of study drug. The PK population comprised safety population participants for which data were available for derivation of ≥1 PK parameter. Pharmacokinetic parameters were calculated by noncompartmental analysis using Phoenix WinNonlin version 8.1 and were summarized by treatment group. Descriptive statistics are presented for plasma concentrations, amount and cumulative amount of Pudexacianinium excreted in urine, and Pudexacianinium concentrations for point urine collection by treatment group and scheduled sample time.

### Results:

There were 30 participants, of whom 20 (4 per cohort) received Pudexacianinium chloride and 10 (2 per cohort) received placebo. All 30 participants completed the study in accordance with the protocol, and all were included in the safety population. All 20 participants receiving Pudexacianinium chloride were included in the PK population. Within each cohort and overall, 50% of participants were female (Table 1). The overall mean age was 43 (standard deviation [SD], 11.3 years), mean BMI was 27.3 kg/m² (SD, 2.96), and 66.7% were white. Mean age and BMI were similar across all cohorts.

TABLE 1

| | | | Pudexacianinium | | | | | | | | | | | |
|---|---|---|---|---|---|---|---|---|---|---|---|---|---|---|
| | Placebo | | 0.1 mg | | 0.5 mg | | 2 mg | | 8 mg | | 24 mg | | Overall | |
| | (n = 10) | | (n = 4) | | (n = 4) | | (n = 4) | | (n = 4) | | (n = 4) | | (N = 30) | |
| Age, mean (SD) | 44 | (11.2) | 43 | (13.2) | 45 | (15.2) | 44 | (11.0) | 43 | (10.7) | 41 | (14.2) | 43 | (11.3) |
| years | | | | | | | | | | | | | | |
| Sex, n (%) | | | | | | | | | | | | | | |
| Female | 5 | (50.0) | 2 | (50.0) | 2 | (50.0) | 2 | (50.0) | 2 | (50.0) | 2 | (50.0) | 15 | (50.0) |
| Male | 5 | (50.0) | 2 | (50.0) | 2 | (50.0) | 2 | (50.0) | 2 | (50.0) | 2 | (50.0) | 15 | (50.0) |
| Race | | | | | | | | | | | | | | |
| White | 7 | (70.0) | 2 | (50.0) | 3 | (75.0) | 2 | (50.0) | 2 | (50.0) | 4 | (100.0) | 20 | (66.7) |
| Black or African | 3 | (30.0) | 2 | (50.0) | 0 | | 2 | (50.0) | 1 | (25.0) | 0 | | 8 | (26.7) |
| American | | | | | | | | | | | | | | |
| Other^{a} | 0 | | 0 | | 1 | (25.0) | 0 | | 1 | (25.0) | 0 | | 2 | (6.6) |
| Hispanic or Latino, | 4 | (40.0) | 0 | | 2 | (50.0) | 1 | (25.0) | 1 | (25.0) | 1 | (25.0) | 9 | (30.0) |
| n (%) | | | | | | | | | | | | | | |
| Weight, mean (SD) | 76.7 | (13.5) | 82.8 | (18.0) | 75.5 | (11.2) | 78.4 | (15.6) | 78.5 | (11.1) | 77.7 | (16.4) | 78.0 | (13.2) |
| kg | | | | | | | | | | | | | | |
| BMI, mean (SD) | 26.6 | (3.5) | 29.0 | (2.9) | 28.0 | (1.1) | 26.5 | (4.2) | 26.9 | (2.8) | 27.5 | (2.2) | 27.3 | (3.0) |
| kg/m² | | | | | | | | | | | | | | |

^{a}Includes Asian and Native Hawaiian or Pacific Islander.
BMI, body mass index; SD, standard deviation.

### Safety and Tolerability:

Overall, treatment-emergent AEs (TEAEs) during the study were reported in 3 participants (15.0%) who received Pudexacianinium chloride and 2 participants (20.0%) who received placebo. Infusions did not lead to histamine release or hypersensitivity. Among participants receiving Pudexacianinium chloride, 1 receiving the 0.5-mg dose of Pudexacianinium as free form experienced oral herpes and presyncope (both grade 1), 1 receiving the 8-mg dose of Pudexacianinium as free form experienced a grade 1 urinary tract infection, and 1 receiving the 24-mg dose experienced grade 1 headache, grade 2 dysuria, and grade 3 pyelonephritis (Table 2).

TABLE 2
Treatment Emergent Adverse Events

| | | Pudexacianinium | | | | | | |
|---|---|---|---|---|---|---|---|---|
| | Placebo | 0.1 mg | 0.5 mg | 2 mg | 8 mg | 24 mg | Overall^{a} | |
| | (n = 10) | (n = 4) | (n = 4) | (n = 4) | (n = 4) | (n = 4) | (N = 20) | |
| Total, n (%) | 2 (20.0) | 0 | 1 (25.0) | 0 | 1 (25.0) | 1 (25.0) | 3 | (15.0) |
| Oral herpes | 0 | 0 | 1 (25.0) | 0 | 0 | 0 | 1 | (5.0) |
| Pyelonephritis | 0 | 0 | 0 | 0 | 0 | 1 (25.0) | 1 | (5.0) |
| Urinary tract | 0 | 0 | 0 | 0 | 1 (25.0) | 0 | 1 | (5.0) |
| infection | | | | | | | | |
| Headache | 0 | 0 | 0 | 0 | 0 | 1 (25.0) | 1 | (5.0) |
| Presyncope | 0 | 0 | 1 (25.0) | 0 | 0 | 0 | 1 | (5.0) |
| Dysuria | 0 | 0 | 0 | 0 | 0 | 1 (25.0) | 1 | (5.0) |

| | | | | | | | |
|---|---|---|---|---|---|---|---|
| Incontinence | 1 (10.0) | 0 | 0 | 0 | 0 | 0 | 0 |
| Nausea | 1 (10.0) | 0 | 0 | 0 | 0 | 0 | 0 |
| Vomiting | 1 (10.0) | 0 | 0 | 0 | 0 | 0 | 0 |

^{a}In participants who received Pudexacianinium (any dose).

No TEAE was considered by investigators to be related or possibly related to Pudexacianinium chloride. Participants who experienced oral herpes, urinary tract infection, and pyelonephritis received corrective medication.

The case of grade 3 pyelonephritis was a serious AE that began on day 2 following Pudexacianinium chloride administration and was attributed to the urethral catheter. This participant was admitted to the hospital on day 3 and treated with ceftriaxone. On day 6 the participant was discharged after switching to oral Bactrim, pyridium, and ibuprofen, at which point the serious AE was considered resolved. There were no clinically significant results or trends in serum chemistry data, hematology data, vital sign measurements, ECG parameters, or physical examinations, nor were there any withdrawals from the study due to AEs or on-study deaths.

### Pharmacokinetics:

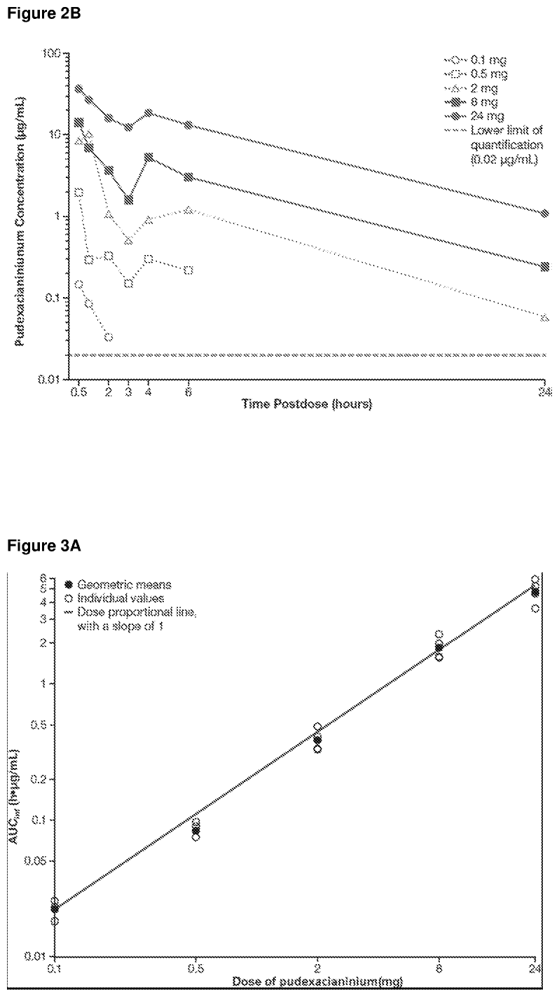

Pudexacianinium arithmetic mean plasma concentrations are shown in FIG. 2A.

The mean terminal half-life of Pudexacianinium ranged from 2.1 to 3.6 hours, and total body clearance and terminal volume of distribution were consistent across the dose range (Table 3).

TABLE 3
Summary of Plasma and Urine PK Parameters for
Pudexacianinium Following Single IV Bolus Doses

| Pudexacianinium | | | | |
|---|---|---|---|---|
| 0.1 mg | 0.5 mg | 2 mg | 8 mg | 24 mg |
| (n = 4) | (n = 4) | (n = 4) | (n = 4) | (n = 4) |

Key Urine PK Parameters

| | | | | | |
|---|---|---|---|---|---|
| Aeₗₐₛₜ (mg) | 0.0768 | 0.403 | 1.68 | 8.01 | 23.1 |
| | (0.0214) | (0.0128) | (0.171) | (0.337) | (1.09) |
| Ae_{last %} (%) | 76.8 | 80.6 | 84.1 | 100 | 96.3 |
| | (21.4) | (2.56) | (8.55) | (4.21) | (4.53) |
| CL_{R} (L/h) | 3.57 | 4.85 | 4.45 | 4.43 | 4.92 |
| | (1.44) | (0.626) | (1.18) | (0.973) | (1.01) |

Key Plasma PK Parameters

| | | | | | |
|---|---|---|---|---|---|
| AUC₀₋₂₄ | 0.0223 | 0.0842 | 0.392 | 1.87 | 4.85 |
| (h · μg/mL) | (0.00309) | (0.0112) | (0.0733) | (0.363) | (0.981) |
| AUC_{inf} | 0.0224 | 0.0843 | 0.392 | 1.88 | 4.87 |
| (h · μg/mL) | (0.00310) | (0.0112) | (0.0734) | (0.370) | (0.985) |
| AUC_{inf} | 19.7 | 6.29 | 5.86 | 0.492 | 0.375 |
| (% extrap)^{a} (%) | (18.1-25.7) | (4.63-7.58) | (4.65-7.78) | (0.327-0.921) | (0.144-0.770) |
| AUCₗₐₛₜ | 0.0178 | 0.0791 | 0.369 | 1.87 | 4.85 |
| (h · μg/mL) | (0.00313) | (0.0110) | (0.0674) | (0.363) | (0.981) |
| Cₘₐₓ (μg/mL) | 0.0161 | 0.0841 | 0.329 | 1.37 | 3.81 |
| | (0.00201) | (0.0199)^{b} | (0.0727) | (0.151) | (1.16) |
| C₀ (μg/mL) | 0.0223 | 0.115 | 0.435 | 1.85 | 4.74 |
| | (0.00235) | (0.0330)^{b} | (0.108) | (0.244) | (1.56) |
| tₘₐₓ^{a} (h) | 0.08 | 0.08 | 0.08 | 0.08 | 0.08 |
| | (0.08-0.17) | (0.08-0.08) | (0.08-0.08) | (0.08-0.08) | (0.08-0.08) |
| tₗₐₛₜ^{a} (h) | 4.00 | 8.00 | 8.00 | 24.00 | 24.03 |
| | (4.00-6.00) | (8.00-8.00) | (8.00-8.02) | (24.00-24.00) | (24.00-24.03) |
| t_{1/2} (h) | 2.24 | 2.23 | 2.10 | 3.58 | 3.41 |
| | (0.380) | (0.0988) | (0.147) | (0.245) | (0.420) |
| CLₜₒₜ (L/h) | 4.54 | 6.01 | 5.22 | 4.38 | 5.10 |
| | (0.687) | (0.789) | (0.918) | (0.820) | (1.12) |
| V_{z} (L) | 14.6 | 19.3 | 15.8 | 22.4 | 25.1 |
| | (2.70) | (2.79) | (2.28) | (2.95) | (6.51) |

Arithmetic means (SD) are presented unless otherwise noted.
^{a}Median (minimum maximum).
^{b}n = 3. Aeₗₐₛₜ, amount of unchanged drug excreted into the urine from time zero to the time of the last quantifiable concentration; Ae_{last %}, percent of unchanged drug excreted into the urine from time zero to the time of the last quantifiable concentration; AUC₀₋₂₄, area under the plasma concentration-time curve from time zero to 24 hours postdose; AUC_{inf}, area under the plasma concentration-time curve from time zero to infinity; AUC_{inf}(% extrap), area under the plasma concentration-time curve extrapolated from time tₗₐₛₜ to infinity as a percentage of total area under the plasma concentration-time curve; AUCₗₐₛₜ, area under the plasma concentration-time curve from time zero to the time of the last quantifiable concentration; C₀, back-extrapolated plasma concentration at time zero; CL_{R}, renal clearance; CLₜₒₜ, total body clearance of drug from plasma; Cₘₐₓ, maximum observed plasma concentration; IV, intravenous; n, number of subjects; PK, pharmacokinetic; SD, standard deviation; t_{1/2}, apparent terminal plasma elimination half-life; tₗₐₛₜ, time of last observed plasma concentration; tₘₐₓ, time of maximum observed plasma concentration; V_{z}, volume of distribution during the terminal phase.

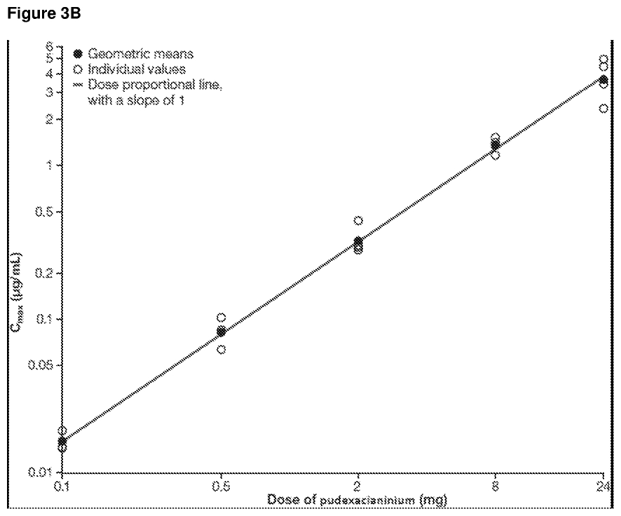

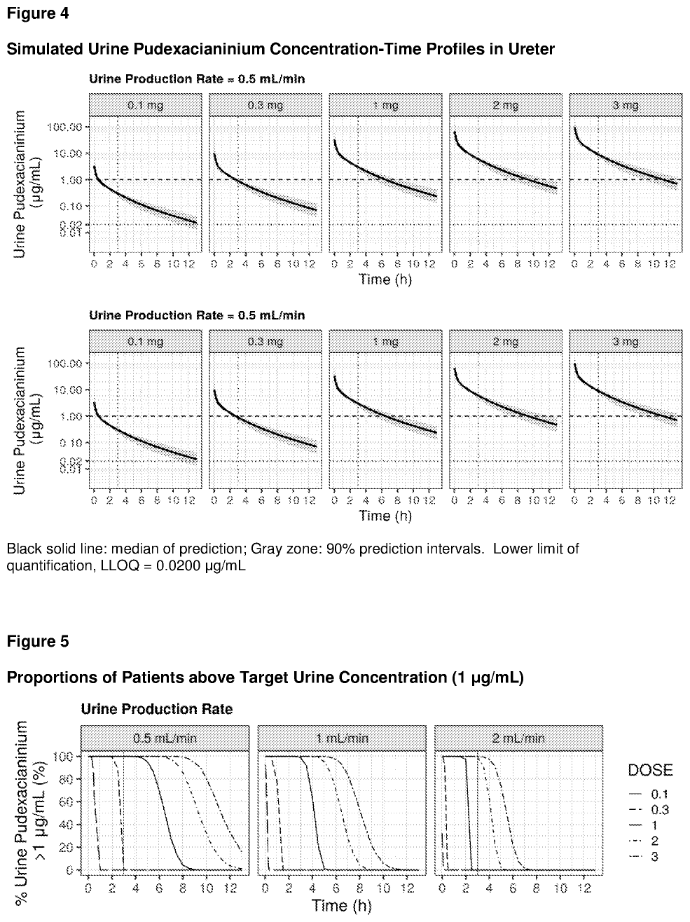

At doses ranging from 0.1 mg to 24 mg of Pudexacianinium as free form, increases in Pudexacianinium AUC_{inf} (FIG. 3A) and Cₘₐₓ (FIG. 3B) were approximately proportional to increases in dose. These dose-proportional increases in exposure coupled with consistent half-life and total body clearance estimates indicate linear PK for Pudexacianinium across the evaluated dose range.

Following IV administration, Pudexacianinium appeared rapidly in the urine, with quantifiable concentrations observed for all participants at the first postdose sample collection point (0.5 hours). Urinary Pudexacianinium concentrations were quantifiable for up to 6 hours postdose in the 0.1-mg and 0.5-mg cohorts, and for up to 24 hours postdose in the 2-mg, 8-mg, and 24-mg cohorts (FIG. 2B). Across all dose ranges, urinary excretion was nearly complete by 24 hours. The mean amount of intact Pudexacianinium recovered in urine was 0.0768, 0.403, 1.68, 8.01, and 23.1 mg following respective Pudexacianinium as free form doses of 0.1, 0.5, 2, 8, or 24 mg. The corresponding percentage of the administered Pudexacianinium dose recovered unchanged in urine (Aeₗₐₛₜ) ranged from 76.8% to 100% (Table 4).

TABLE 4
Summary of Plasma and Urine PK Parameters for
Pudexacianinium Following Single IV Bolus Doses

| Pudexacianinium | | | | |
|---|---|---|---|---|
| 0.1 mg | 0.5 mg | 2 mg | 8 mg | 24 mg |
| (n = 4) | (n = 4) | (n = 4) | (n = 4) | (n = 4) |

Key Urine PK Parameters

| | | | | | |
|---|---|---|---|---|---|
| Aeₗₐₛₜ (mg) | 0.0768 | 0.403 | 1.68 | 8.01 | 23.1 |
| | (0.0214) | (0.0128) | (0.171) | (0.337) | (1.09) |
| Ae_{last %} (%) | 76.8 | 80.6 | 84.1 | 100 | 96.3 |
| | (21.4) | (2.56) | (8.55) | (4.21) | (4.53) |
| CL_{R} (L/h) | 3.57 | 4.85 | 4.45 | 4.43 | 4.92 |
| | (1.44) | (0.626) | (1.18) | (0.973) | (1.01) |

Key Plasma PK Parameters

| | | | | | |
|---|---|---|---|---|---|
| AUC₀₋₂₄ | 0.0223 | 0.0842 | 0.392 | 1.87 | 4.85 |
| (h · μg/mL) | (0.00309) | (0.0112) | (0.0733) | (0.363) | (0.981) |
| AUC_{inf} | 0.0224 | 0.0843 | 0.392 | 1.88 | 4.87 |
| (h · μg/mL) | (0.00310) | (0.0112) | (0.0734) | (0.370) | (0.985) |
| AUC_{inf} | 19.7 | 6.29 | 5.86 | 0.492 | 0.375 |
| (% extrap)^{a} (%) | (18.1-25.7) | (4.63-7.58) | (4.65-7.78) | (0.327-0.921) | (0.144-0.770) |
| AUCₗₐₛₜ | 0.0178 | 0.0791 | 0.369 | 1.87 | 4.85 |
| (h · μg/mL) | (0.00313) | (0.0110) | (0.0674) | (0.363) | (0.981) |
| Cₘₐₓ (μg/mL) | 0.0161 | 0.0841 | 0.329 | 1.37 | 3.81 |
| | (0.00201) | (0.0199)^{b} | (0.0727) | (0.151) | (1.16) |
| C₀ (μg/mL) | 0.0223 | 0.115 | 0.435 | 1.85 | 4.74 |
| | (0.00235) | (0.0330)^{b} | (0.108) | (0.244) | (1.56) |
| tₘₐₓ^{a} (h) | 0.08 | 0.08 | 0.08 | 0.08 | 0.08 |
| | (0.08-0.17) | (0.08-0.08) | (0.08-0.08) | (0.08-0.08) | (0.08-0.08) |
| tₗₐₛₜ^{a} (h) | 4.00 | 8.00 | 8.00 | 24.00 | 24.03 |
| | (4.00-6.00) | (8.00-8.00) | (8.00-8.02) | (24.00-24.00) | (24.00-24.03) |
| t_{1/2} (h) | 2.24 | 2.23 | 2.10 | 3.58 | 3.41 |
| | (0.380) | (0.0988) | (0.147) | (0.245) | (0.420) |
| CLₜₒₜ (L/h) | 4.54 | 6.01 | 5.22 | 4.38 | 5.10 |
| | (0.687) | (0.789) | (0.918) | (0.820) | (1.12) |
| V_{z} (L) | 14.6 | 19.3 | 15.8 | 22.4 | 25.1 |
| | (2.70) | (2.79) | (2.28) | (2.95) | (6.51) |

Arithmetic means (SD) are presented unless otherwise noted.
^{a}Median (minimum maximum).
^{b}n = 3. Aeₗₐₛₜ, amount of unchanged drug excreted into the urine from time zero to the time of the last quantifiable concentration; Ae_{last %}, percent of unchanged drug excreted into the urine from time zero to the time of the last quantifiable concentration; AUC₀₋₂₄, area under the plasma concentration-time curve from time zero to 24 hours postdose; AUC_{inf}, area under the plasma concentration-time curve from time zero to infinity; AUC_{inf}(% extrap), area under the plasma concentration-time curve extrapolated from time tₗₐₛₜ to infinity as a percentage of total area under the plasma concentration-time curve; AUCₗₐₛₜ, area under the plasma concentration-time curve from time zero to the time of the last quantifiable concentration; C₀, back-extrapolated plasma concentration at time zero; CL_{R}, renal clearance; CLₜₒₜ, total body clearance of drug from plasma; Cₘₐₓ, maximum observed plasma concentration; IV, intravenous; n, number of subjects; PK, pharmacokinetic; SD, standard deviation; t_{1/2}, apparent terminal plasma elimination half-life; tₗₐₛₜ, time of last observed plasma concentration; tₘₐₓ, time of maximum observed plasma concentration; V_{z}, volume of distribution during the terminal phase.

As a point of reference, in the preclinical study using a minipig model, Pudexacianinium was totally excreted (95%) within 6 hours after IV administration.²²

Visible green urine coloration occurred in participants who received 8-mg and 24-mg doses of Pudexacianinium as free form, but not in those receiving placebo. In 7 of these 8 participants, green urine coloration was first observed at the initial urine interval collection (0 to 0.5 hours) (Table 5).

TABLE 5
Timing of Green Urine Coloration

| | | | Interval When | Interval When |
|---|---|---|---|---|
| | | | Green | Green Coloration |
| | Order of | | Coloration Was | Was No Longer |
| | Dosing per | | First Observed, | Observed, Hour |
| Cohort | Cohort | Sex | Hour Postdose | Postdose |
| 8 mg | 1 | Male | 0 to 0.5 | 1 to 1.5 |
| | 2 | Male | 0 to 0.5 | 3 to 3.5 |
| | 3 | Female | 2 to 2.5 | 3 to 3.5 |
| | 4 | Female | 0 to 0.5 | 3 to 3.5 |
| 24 mg | 1 | Male | 0 to 0.5 | 8 to 12 |
| | 2 | Female | 0 to 0.5 | 8 to 12 |
| | 3 | Female | 0 to 0.5 | 12 to 24 |
| | 4 | Male | 0 to 0.5 | 12 to 24 |

In the remaining participant (8-mg cohort), green urine coloration first appeared in the 2- to 2.5-hour interval. Green urine was observed until 3 to 3.5 hours postdose in 3 of the 4 participants in the 8-mg cohort, and until 12 to 24 hours postdose in 2 of the 4 participants in the 24-mg cohort. In all cases, green urine coloration was no longer observed by 24 hours postdose.

### Summary of Results of Example 1

There are currently no FDA-approved agents to facilitate intraoperative NIRF visualization of the ureter. Experimental studies of the dyes, methylene blue and indocyanine green, which are approved for use in other indications²³⁻²⁵, have revealed deficiencies in optical properties and route of administration and clearance, respectively, that make them unsuitable for such procedures.¹² To address this unmet need, novel NIRF agents are being developed. Several of these, including IS-001,¹⁷ IRDye® 800-BK,^{26,27} and ZW800-1,²⁸ have recently progressed to first-in-human studies.

Herein provided is first-in-human data for Pudexacianinium chloride, which was designed for high water solubility, low self-aggregation (which can reduce fluorescence), and high optical and chemical stability.²⁰ These results, obtained in healthy volunteers, provide key information regarding the safety and PK profiles of Pudexacianinium, including a dose estimation for a phase 2 study design. Single ascending IV bolus doses of up to 24 mg did not lead to any Pudexacianinium-related TEAEs and there were no withdrawals due to AEs. One serious TEAE, grade 3 pyelonephritis, occurred in 1 participant and, consistent with urology observation spanning decades,^{29, 30} was found to be a complication related to the urethral catheter. No significant trends in serum chemistry data, hematology data, vital sign measurements, ECG parameters, or physical examinations were observed in any participant.

Pharmacokinetic analysis revealed that Pudexacianinium is primarily excreted unchanged into urine, appearing there rapidly, with its excretion nearly complete at 24 hours. A single 8-mg or 24-mg dose afforded noticeable green urine coloration for at least 3 hours in most participants. Linear and dose-proportional Pudexacianinium plasma PK were observed across the evaluated dose range.

In conclusion, based on preclinical results demonstrating that IV Pudexacianinium chloride allows distinct NIRF ureter visualization at 0.01 mg/kg²², the Pudexacianinium urine concentrations and PK parameters found in this study support intraoperative NIRF ureter visualization using Pudexacianinium as free form within the 0.1-mg to 24-mg dose range. Collectively, these safety and PK results support further evaluation of Pudexacianinium chloride for ureter detection during surgical procedures of the abdomen and pelvis.

### Example 2—Clinical Dose Setting of Pudexacianinium Chloride

Near-infrared fluorescence (NIR-F) ureter visualization depends on the urine concentration of Pudexacianinium in the ureter. It is likely that the (real-time) urine concentration of Pudexacianinium can be a good pharmacodynamic (visualization) surrogate marker. Ureter imaging studies in minipigs suggested sufficient urine Pudexacianinium concentration in the urinary tract would be 1 μg/mL for NIR-F ureter imaging based on ex vivo test using a ureter of the pigs (Mol Imaging Biol (2021)).

The target product profile of intraoperative ureter imaging by Pudexacianinium chloride is that almost all patients achieve clear ureter visualization during surgery (for 3 hours after IV bolus administration).

Therefore, a target dose which achieve urine concentrations over 1 μg/mL for 3 hours after dose was explored. In the phase 1 healthy volunteer study (ClinicalTrials.gov Identifier: NCT03698305), mean point urine concentration of Pudexacianinium at 3 hours after dose of 2 mg IV bolus was 0.5 μg/mL under no water intake restriction.

In general, urine drug concentrations in humans are highly variable because the concentration is affected by urine formation volume (urine output). Actually, in the US phase 1 study (ClinicalTrials.gov Identifier: NCT03698305) observed urine concentrations in healthy volunteers were highly variable and affected by the urine volume variation. In patients under anesthesia during surgery, it is recommended to control the urine production rate at low level around 1 mL/min [Davison A and Ross J A, 2016³¹ and Puckett et al., 2017³²] while in the US phase 1 study participants consumed water ad libitum throughout the study and large variability in urine concentration was observed. In order to exactly estimate the clinical dose of Pudexacianinium for patients under anesthesia during surgery, it was considered necessary to use a pharmacological mechanism based quantitative approach.

A population pharmacokinetic model was developed by nonlinear mixed effects modeling using the data from the US phase 1 healthy volunteer study. A 3-compartment model well described the plasma concentration-time profiles of Pudexacianinium, and urine Pudexacianinium concentrations were simultaneously analyzed using output compartment. The model succeeded to describe the individual urine Pudexacianinium concentration. Using the developed model, plasma and urine Pudexacianinium concentration-time profiles in individual patients under anesthesia during surgery were simulated for the clinical dose setting. The target product profile of intraoperative ureter imaging by Pudexacianinium is that almost all patients achieve clear ureter visualization during surgery (for 3 hours after IV bolus administration).

The simulation were performed assuming the following 3 points that were all considered important:
(1) urine production rate under surgery would be controlled at 1 mL/min,
(2) no inter-individual variability in the urine production rate, and
(3) no delay between plasma concentration and excretion into urine.

A total of 1000 virtual patient population was generated to simulate plasma and urine Pudexacianinium concentration-time profiles for each dose (0.1, 0.3, 1, 2, and 3 mg). The simulations were sequentially performed including 2 mg which showed the mean urine concentrations close to the target concentration in the phase 1 study. The simulated urine Pudexacianinium concentration-time courses in patients under anesthesia during surgery are presented in Error! Reference source not found..

The impact of change in urine production rate was also tested ranging from 0.5 to 2 mL/min. The simulated proportions of patients above target urine concentration (1 μg/mL) suggested that 1 mg administration to the patients with urine production rate from 0.5 to 1.0 mL/min would achieve urine concentrations over 1 μg/mL for 3 hours after single IV dose in more than 99% patients (Error! Reference source not found.).

Three dose levels of 0.3, 1 and 3 mg per participant were proposed as initial doses in the phase 2 study by selecting 1 mg as the central dose and using a common ratio of “3” to differentiate between doses.

### REFERENCES

1. Dobrowolski Z, Kusionowicz J, Drewniak T, et al. Renal and ureteric trauma: diagnosis and management in Poland. BJU Int. 2002; 89(7):748-751.
2. Ahn C B, Kim J H, Park G K, et al. Prognostic imaging of iatrogenic and traumatic ureteral injury by near-infrared fluorescence. Quant Imaging Med Surg. 2019; 9(6):1056-1065.
3. Summerton D J, Kitrey N D, Lumen N, Serafetinidis E, Djakovic N, European Association of U. EAU guidelines on iatrogenic trauma. Eur Urol. 2012; 62(4):628-639.
4. Abboudi H, Ahmed K, Royle J, Khan M S, Dasgupta P, N'Dow J. Ureteric injury: a challenging condition to diagnose and manage. Nat Rev Urol. 2013; 10(2):108-115.
5. Halabi W J, Jafari M D, Nguyen V Q, et al. Ureteral injuries in colorectal surgery: an analysis of trends, outcomes, and risk factors over a 10-year period in the United States. Dis Colon Rectum. 2014; 57(2):179-186.
6. Lucarelli G, Ditonno P, Bettocchi C, et al. Delayed relief of ureteral obstruction is implicated in the long-term development of renal damage and arterial hypertension in patients with unilateral ureteral injury. J Urol. 2013; 189(3):960-965.
7. Nachiappan S C A, Askari A, Faiz O. Intraoperative ureteric injuries and litigation in the NHS. J Clin Urol. 2015; 8(1):9-13.
8. Brandes S, Coburn M, Armenakas N, McAninch J. Diagnosis and management of ureteric injury: an evidence-based analysis. BJU Int. 2004; 94(3):277-289.
9. Tan-Kim J, Menefee S A, Reinsch C S, et al. Laparoscopic Hysterectomy and Urinary Tract Injury: Experience in a Health Maintenance Organization. J Minim Invasive Gynecol. 2015; 22(7):1278-1286.
10. Parpala-Sparman T, Paananen I, Santala M, Ohtonen P, Hellstrom P. Increasing numbers of ureteric injuries after the introduction of laparoscopic surgery. Scand J Urol Nephrol. 2008; 42(5):422-427.
11. de Valk K S, Handgraaf H J, Deken M M, et al. A zwitterionic near-infrared fluorophore for real-time ureter identification during laparoscopic abdominopelvic surgery. Nat Commun. 2019; 10(1):3118.
12. Slooter M D, Janssen A, Bemelman W A, Tanis P J, Hompes R. Currently available and experimental dyes for intraoperative near-infrared fluorescence imaging of the ureters: a systematic review. Tech Coloproctol. 2019; 23(4):305-313.
13. Wallis C J, Cheung D C, Garbens A, et al. Occurrence of and Risk Factors for Urological Intervention During Benign Hysterectomy: Analysis of the National Surgical Quality Improvement Program Database. Urology. 2016; 97:66-72.
14. Douissard J, Meyer J, Ris F, Liot E, Morel P, Buchs N C. latrogenic ureteral injuries and their prevention in colorectal surgery: results from a nationwide survey. Colorectal Dis. 2019; 21(5):595-602.
15. Lynch T H, Martinez-Pineiro L, Plas E, et al. EAU guidelines on urological trauma. Eur Urol. 2005; 47(1):1-15.
16. AI-Taher M, van den Bos J, Schols R M, Bouvy N D, Stassen L P. Fluorescence Ureteral Visualization in Human Laparoscopic Colorectal Surgery Using Methylene Blue. J Laparoendosc Adv Surg Tech A. 2016; 26(11):870-875.
17. Farnam R W, Arms R G, Klaassen A H, Sorger J M. Intraoperative ureter visualization using a near-infrared imaging agent. J Biomed Opt. 2019; 24(6):1-8.
18. Hyun H, Henary M, Gao T, et al. 700-nm Zwitterionic Near-Infrared Fluorophores for Dual-Channel Image-Guided Surgery. Mol Imaging Biol. 2016; 18(1):52-61.
19. Verbeek F P, van der Vorst J R, Schaafsma B E, et al. Intraoperative near infrared fluorescence guided identification of the ureters using low dose methylene blue: a first in human experience. J Urol. 2013; 190(2):574-579.
20. Kurahashi T, Iwatsuki K, Onishi T, Arai T, Teranishi K, Hirata H. Near-infrared indocyanine dye permits real-time characterization of both venous and lymphatic circulation. J Biomed Opt. 2016; 21(8):86009.
21. Gioux S, Choi H S, Frangioni J V. Image-guided surgery using invisible near-infrared light: fundamentals of clinical translation. Mol Imaging. 2010; 9(5):237-255.
22. Fushiki H, Yoshikawa T, Matsuda T, Sato T, Suwa A. Preclinical Development and Validation of ASP5354: A Near-Infrared Fluorescent Agent for Intraoperative Ureter Visualization. Molecular Imaging and Biology. 2021; May 11 online ahead of print.
23. Carr J A, Franke D, Caram J R, et al. Shortwave infrared fluorescence imaging with the clinically approved near-infrared dye indocyanine green. Proc Natl Acad Sci USA. 2018; 115(17):4465-4470.
24. Liu Y, Jia Q, Zhou J. Recent Advance in Near-Infrared (NIR) Imaging Probes for Cancer Theranostics. Advanced Therapeutics. 2018; 1(8):1800055.
25. Said S M, Marey G, Hiremath G. Intraoperative fluorescence with indocyanine green in congenital cardiac surgery: Potential applications of a novel technology. JTCVS Techniques. 2021.
26. AI-Taher M, van den Bos J, Schols R M, Kubat B, Bouvy N D, Stassen L P S. Evaluation of a novel dye for near-infrared fluorescence delineation of the ureters during laparoscopy. BJS Open. 2018; 2(4):254-261.
27. van den Bos J, AI-Taher M, Bouvy N D, Stassen L P S. Near-infrared fluorescence laparoscopy of the ureter with three preclinical dyes in a pig model. Surg Endosc. 2019; 33(3):986-991.
28. Choi H S, Nasr K, Alyabyev S, et al. Synthesis and in vivo fate of zwitterionic near-infrared fluorophores. Angew Chem Int Ed Engl. 2011; 50(28):6258-6263.
29. Prather G C, Sears B R. Pyelonephritis: in defense of the urethral catheter. J Urol. 1960; 83:337-344.
30. Cortese Y J, Wagner V E, Tierney M, Devine D, Fogarty A. Review of Catheter-Associated Urinary Tract Infections and In Vitro Urinary Tract Models. J Healthc Eng. 2018; 2018:2986742.
31. Davison A, Ross J A. Chapter 55 Patient with poor urine output. In: Coomarasamy A. Shafi M I, Davila G W, Chan K K, editors. Gynecologic and obstetric surgery: challenges and management options. John Wiley & Sons Inc. (NY): 2016; 167-9
32. Puckett J R, Pickering J W, Palmer S C, McCall J L, Kluger M T, De Zoysa J, et al. Low versus standard urine output targets in patients undergoing major abdominal surgery: A Randomized Noninferiority Trial. Ann Surg. 2017; 265(5):874-81.

1. A pharmaceutical composition comprising Pudexacianinium or a pharmaceutically acceptable salt thereof for imaging at least one of an organ, a body fluid, and a vessel in a subject by near-infrared fluorescence (NIRF), wherein said pharmaceutical composition comprises 0.3 mg to 24.0 mg of Pudexacianinium, based upon a free form weight, and said pharmaceutical composition is administered to the subject in a first administration and optionally said pharmaceutical composition comprising 0.3 mg to 24.0 mg of Pudexacianinium, based upon a free form weight, is re-administered to subject after the first administration.

2. The pharmaceutical composition according to claim 1, wherein said pharmaceutical composition comprising 0.3 mg to 24.0 mg of Pudexacianinium, based upon a free form weight, is re-administered to subject after the first administration.

3. The pharmaceutical composition according to claim 1, wherein said pharmaceutical composition comprises 0.3 mg to 3.0 mg of Pudexacianinium, based upon a free form weight.

4. The pharmaceutical composition according to claim 1, wherein said pharmaceutical composition comprises 1.0 mg to 3.0 mg of Pudexacianinium, based upon a free form weight.

5. The pharmaceutical composition according to claim 1, wherein Pudexacianinium or a pharmaceutically acceptable salt thereof is Pudexacianinium chloride.

6. A method for imaging at least one of an organ, a body fluid, and a vessel in a subject by near-infrared fluorescence (NIRF) comprising
administering to the subject a first pharmaceutical composition comprising Pudexacianinium or a pharmaceutically acceptable salt thereof at a dosage of 0.3 mg to 24.0 mg of Pudexacianinium, based upon a free form weight;
subjecting the subject to NIRF to obtain an image of the at least one of an organ, a body fluid, and a vessel in the subject; and
optionally administering to the subject a subsequent pharmaceutical composition comprising Pudexacianinium or a pharmaceutically acceptable salt thereof at a dosage of 0.3 mg to 24.0 mg of Pudexacianinium, based upon a free form weight.

7. The method of claim 6, wherein the first pharmaceutical composition comprises Pudexacianinium or a pharmaceutically acceptable salt thereof at a dosage of 0.3 mg to 3.0 mg of Pudexacianinium, based upon a free form weight, or the subsequent pharmaceutical composition comprises Pudexacianinium or a pharmaceutically acceptable salt thereof at a dosage of 0.3 mg to 3.0 mg of Pudexacianinium, based upon a free form weight, or both.

8. The method of claim 6, wherein the first pharmaceutical composition comprises Pudexacianinium or pharmaceutically acceptable salt thereof at a dosage of 1.0 mg to 3.0 mg of Pudexacianinium, based upon a free form weight, or the subsequent pharmaceutical composition comprises Pudexacianinium or pharmaceutically acceptable salt thereof at a dosage of 1.0 mg to 3.0 mg of Pudexacianinium, based upon a free form weight, or both.

9. The method according to claim 6, wherein Pudexacianinium or pharmaceutically acceptable salt thereof is Pudexacianinium chloride.

10.-13. (canceled)

14. The pharmaceutical composition according to claim 1, wherein the administration is intravenous administration.

15. The pharmaceutical composition according to claim 1, wherein the imaging is ureter imaging.

16. The pharmaceutical composition according to claim 1, wherein
the pharmaceutical composition comprises 1.0 mg to 3.0 mg Pudexacianinium, based upon a free form weight;
the administration is intravenous administration,
the imaging is ureter imaging, and
Pudexacianinium or a pharmaceutically acceptable salt thereof is Pudexacianinium chloride.

17. The method according to claim 6, wherein the administration is intravenous administration.

18. The method according to claim 6, wherein the imaging is ureter imaging.

19. The method according to claim 6, wherein
the first pharmaceutical composition comprises Pudexacianinium or pharmaceutically acceptable salt thereof at a dosage of 1.0 mg to 3.0 mg of Pudexacianinium, based upon a free form weight, or the subsequent pharmaceutical composition comprises Pudexacianinium or pharmaceutically acceptable salt thereof at a dosage of 1.0 mg to 3.0 mg of Pudexacianinium, based upon a free form weight, or both,
the administration is intravenous administration;
the imaging is ureter imaging, and
Pudexacianinium or a pharmaceutically acceptable salt thereof is Pudexacianinium chloride.
